(19) United States
(12) Patent Application Publication
(10) Pub. No.: US 2021/0307736 A1
(43) Pub. Date: Oct. 7, 2021

(54) METHOD FOR TREATING ALOPECIA

(71) Applicant: Show-Chwan Memorial Hospital, Changhua City (TW)

(72) Inventor: Ming-Chou Ku, Lukang Township (TW)

(21) Appl. No.: 16/837,374

(22) Filed: Apr. 1, 2020

(51) Int. Cl. A61B 17/00

(52) U.S. Cl. CPC A61B 17/00234; A61B 2017/00349

(57) ABSTRACT

A method for treating alopecia is disclosed. The method includes embedding a plurality of first implants and a plurality of second implants in subcutaneous tissue of scalp in accordance with an arrangement, each of the first implant and the second implant comprising a body and a plurality of hooks on the body, wherein in the said arrangement, each of the plurality of hooks on the plurality of first implants and the plurality of second implants is pointing upward.

### FIELD

The exemplary embodiment(s) of the present invention relates to a method for treating alopecia. More specifically, the exemplary embodiment(s) of the present invention relates to a method for treating alopecia by pulling upward a scalp which suffers from alopecia.

### BACKGROUND

Alopecia indicates hair loss or baldness which may affect just your scalp or your entire body. Alopecia may be a result of heredity, hormonal changes, health related, medical conditions, medications or gravity.

Theory that the gravity is partly responsible for hair loss believes that gravity pulls down the skin on the scalp and applies pressure on the scalp, so that causing hair loss. The gravity pulls the scalp downward from the top of a head everyday as long as one not lying in bed and puts pressure over blood vessels in a subcutaneous tissue under the stretched scalp over the top of the head. As the age increases, the pressure applied onto the hair follicles increases, the hair follicles are more pressed between the scalp and the cranial bones and the hair follicles have to work harder to against the pressure. It compromises a circulation of the scalp at the top of the head which nourishes hair follicles. No wonder that we have observed the scalp at the top of the head becomes much thinner on the people after middle age. The hair follicle may wither through the life time as the subcutaneous tissue atrophied through time. The process can be witness by the thickness and tightness of scalp at the top of the head, the tighter the scalp, the less the hair.

The hair follicle is rooted in the subcutaneous tissue under a dermis layer, received nutrition from blood vessels in the subcutaneous tissue, and needs good circulation. Without enough space, the hair follicle and scalp could not get enough nutrients for circulation. Most of treatments to stimulate hair growth are trying to improve the circulation of the scalp through oral or topical medication, such as finasteride (Propecia), dutasteride (Avodart), Minoxidil and spironolactone, even injection of growth factors, such as PRP (Platelet-Rich-Plasma). However, those conventional methods have little effective treatment for hair loss because there is no enough space for the hair follicles, and the pressure is still applied onto the hair follicles. Accordingly, there is a need to develop a method to treat alopecia effectively.

### SUMMARY

The present invention has been made in an effort to find a way to stop hair loss by pulling upward a scalp which suffers from alopecia to against the gravity.

To achieve the above object, a method for treating alopecia according to the present invention is provided. The method for treating alopecia comprises embedding a plurality of first implants and a plurality of second implants in subcutaneous tissue of scalp in accordance with an arrangement, wherein in the said arrangement: directions of the plurality of the first implants are from one side temporal area of a head to the top of the head; directions of the plurality of the second implants are from another side temporal area of the head to the top of the head; the plurality of the first implants and the plurality of the second implants are substantially in parallel with a horizontal line of the top of the head; each of the plurality of the first implants is bonded with a corresponding second implant of the plurality of the second implants at the top of the head to form a plurality pairs of implants; and each of the plurality of hooks on the plurality of the first implants and the plurality of the second implants is pointing upward.

In various embodiments, the body of each of the first implant and the second implant may be a ribbon.

In various embodiments, the body of each of the first implant and the second implant may be a suture.

In various embodiments, the number of the plurality pairs of the implants may be 4-10.

In various embodiments, a space between adjacent one of the plurality pairs of the implants may be substantially 1.5-2 cm.

In various embodiments, the method may further comprise injecting growth factors into the subcutaneous tissue of the scalp.

In various embodiments, the method may further comprise embedding a third implant in the subcutaneous tissue of the scalp, wherein the third implant has a ring-shaped body and a plurality of spikes on the ring-shaped body, and the ring-shaped body encloses the top of the head and intersects with the plurality of the first implants and the plurality of the second implants respectively.

In various embodiments, an angle between each of the plurality of the spikes and the ring-shaped body may be substantially 90°.

In various embodiments, a diameter of the ring-shaped body may be substantially 10-20 cm.

In various embodiments, the ring-shaped body may be a ring-shaped ribbon.

With the above arrangements, the method for treating alopecia according to the present invention has one or more of the advantages of:

(1) increasing a space under the scalp by pulling the skin upward;

(2) improving thickness of the scalp;

(3) improving scalp blood flow of the scalp; and

(4) enlarging hair diameter of the hair on the scalp.

With these and other objects, advantages, and features of the invention that may become hereinafter apparent, the nature of the invention may be more clearly understood by reference to the detailed description of the invention, the embodiments and to the several drawings herein.

### BRIEF DESCRIPTION OF THE DRAWINGS

The exemplary embodiment(s) of the present invention will be understood more fully from the detailed description given below and from the accompanying drawings of various embodiments of the invention, which, however, should not be taken to limit the invention to the specific embodiments, but are for explanation and understanding only.

FIG. 1 is a schematic view illustrating an arrangement according to an exemplary embodiment of the present invention.

FIG. 2 is a schematic view illustrating how to embed the implant in accordance with the arrangement of FIG. 1 according to an exemplary embodiment of the present invention.

FIG. 3 is a schematic view of a first implant according to an exemplary embodiment of the present inventive concept.

FIG. 4 is a schematic view of a first implant according to the other exemplary embodiment of the present inventive concept.

FIG. 5 is a schematic view illustrating an arrangement according to the other exemplary embodiment of the present invention.

FIG. 6 is a schematic view of a third implant of FIG. 5 according to an exemplary embodiment of the present invention.

FIG. 7 is a schematic view illustrating distribution of areas I-IV of an example of the present invention.

FIG. 8 is a high-resolution ultrasound image of the scalp of areas I˜IV of the example of the present invention.

FIG. 9 is a full-field laser perfusion image of the scalp of areas I˜IV of the example of the present invention.

FIG. 10 is an optical microscope image of the scalp of areas I˜IV of the example of the present invention.

### DETAILED DESCRIPTION

Reference will now be made in detail to example embodiments of the present invention, examples of which are illustrated in the accompanying drawings, wherein like reference numerals refer to like elements throughout. In this regard, the present embodiments may have various forms and should not be construed as being limited to the descriptions set forth herein. Accordingly, the embodiments are merely described below, by reference to the figures, to explain aspects of the present description.

The present invention may be variously modified and may have several embodiments. Accordingly, embodiments will be illustrated in the drawings and described in the detailed description as examples only. The effects and features of the present invention, and implementation methods thereof, may be clarified through the description of the following embodiments with reference to the accompanying drawings. The present invention may, however, be embodied in various different forms and should not be construed as limited to the embodiments set forth herein.

Embodiments of the present invention are described below in detail with reference to the accompanying drawings, and when referring to the drawings, the same or similar components are denoted by the same reference numerals and are not repetitively described.

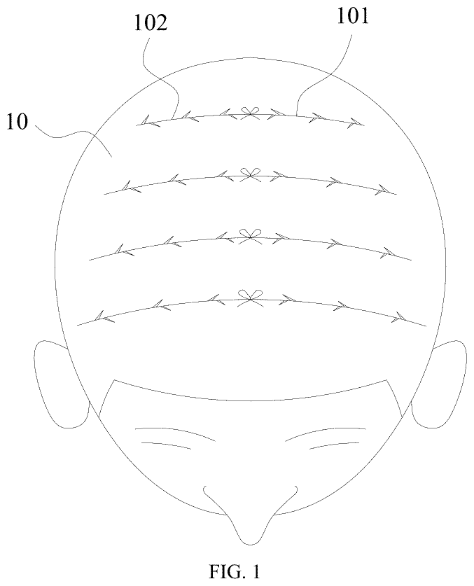

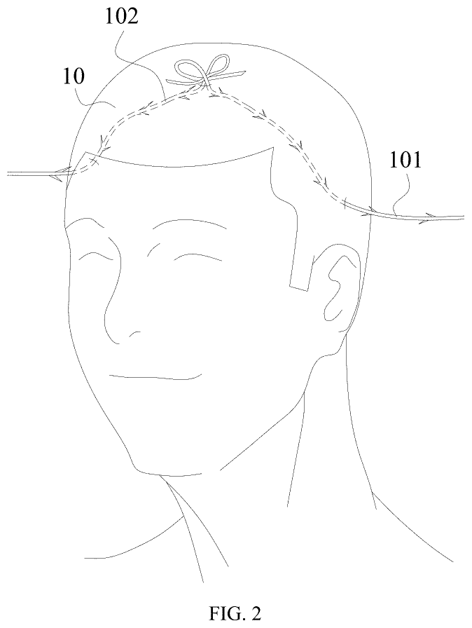

The present invention provides a method for treating alopecia, comprising embedding a plurality of first implants and a plurality of second implants in subcutaneous tissue of scalp in accordance with an arrangement. FIG. 1 is a schematic view illustrating an arrangement according to an exemplary embodiment of the present invention and FIG. 2 is a schematic view illustrating how to embed a first implant 101 and a second implant 102 in accordance with the arrangement of FIG. 1 according to an exemplary embodiment of the present invention.

Referring to FIG. 1, each of the first implant 101 and the second implant 102 comprises a body and a plurality of hooks on the body, and in the arrangement according to an exemplary embodiment of the present invention: directions of the plurality of the first implants 101 are from one side temporal area of a head to the top of the head 10; directions of the plurality of the second implants 102 are from another side temporal area of the head to the top of the head; the plurality of the first implants 101 and the plurality of the second implants 102 are substantially in parallel with a horizontal line of the top of the head 10; each of the plurality of the first implants 101 is bonded with a corresponding second implant of the plurality of the second implants 102 at the top of the head 10 to form a plurality pairs of implants; and each of the plurality of the hooks on the plurality of the first implants 101 and the plurality of the second implants 102 is pointing upward. The expression “a horizontal line of the top of the head” used herein refers to a line passing through the one side temporal area of the head, the top of the head, and the another side temporal area of the head.

Referring to FIG. 2, an end of the first implant 101 may be first inserted into the subcutaneous tissue in the scalp at a first point in one side temporal area of the head 10 and out of the subcutaneous tissue in the scalp at a second point at the top of the head 10, an end of the second implant 102 may be inserted into the subcutaneous tissue in the scalp at a third point in another side temporal area of the head 10 and out of the subcutaneous tissue in the scalp at the second point at the top of the head 10, and then the first implant 101 could be bonded with the second implant 102 at the second point at the top of the head to form a pair of the implants. The embed operation mentioned above may be repeated many times to embed a desired number of the pairs of the implants in the subcutaneous tissue in the scalp in needed thereof. Referring back to FIG. 1, four pairs of the implants are presented in FIG. 1 for illustrating an arrangement according to an exemplary embodiment of the present invention, but exemplary embodiments are not so limited. The number of the pairs of the implants may be 4-10. If the number of the pairs of the implants is less than 4, the force to pull the scalp from the temporal areas toward to the top of the head 10 may be too small to against the gravity. If the number of the pairs of the implants is more than 10, it may lead poor circulation of the scalp. In the arrangement, a space between two adjacent pairs of the implants is substantially 1.5-2 cm, but exemplary embodiments are not so limited. If the space between the two adjacent pairs of the implants is larger than 2 cm, the force to pull the scalp from the temporal areas toward to the top of the head 10 may be too small to against the gravity. If the space between the two adjacent pairs of the implants is smaller than 1.5 cm, it may lead poor circulation of the scalp. In an embodiment, the method of the present invention further comprises injecting growth factors into the subcutaneous tissue of the scalp to stimulate the rejuvenation process.

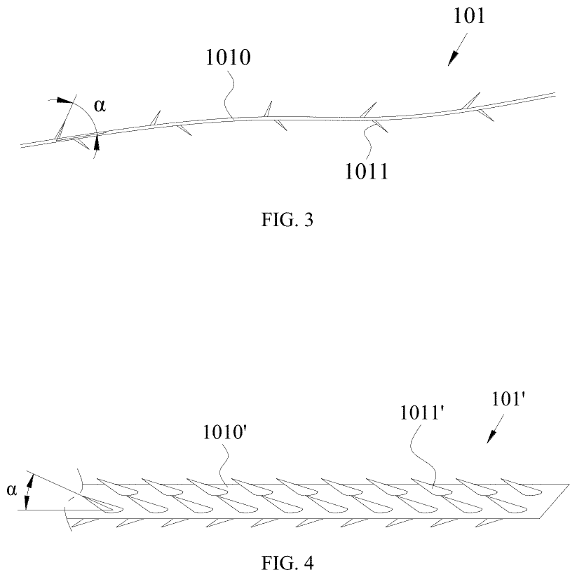

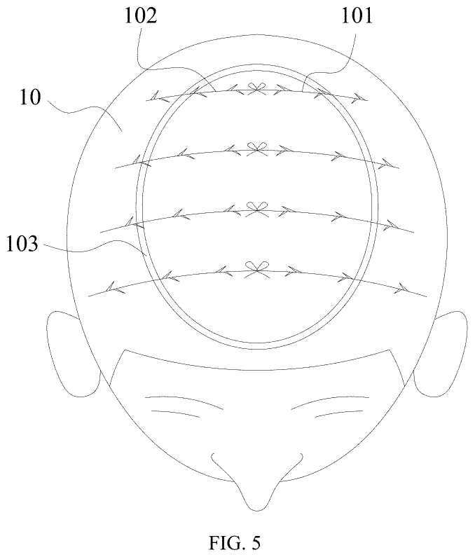

The second implant is substantially the same as the first implant. Therefore, even though the following provides only the structure of the first implant as an example, it is apparent that the descriptions of the first implant could be applied to the second implant too. FIG. 3 is a schematic view of the first implant 101 according to an exemplary embodiment of the present invention and FIG. 4 is a schematic view of a first implant 101′ according to the other exemplary embodiment of the present inventive concept. Referring to FIG. 3, the first implant 101 according to an exemplary embodiment of the present invention has a body 1010 and a plurality of hooks 1011 which can engage dermis and pulling scalp in needed thereof from the sides of skull upward (i.e. pulling the scalp from one side temporal area toward to a zenith) on the body 1010. The body 1010 of the first implant 101 according to an exemplary embodiment of the present invention may be a suture made of non-absorbable materials or absorbable materials. The example of the non-absorbable materials may include but not limit to nylon, polypropylene (Prolene), polybutester (Novafil), and any combination thereof. The example of the absorbable materials may include but not limit to catgut; polyglycolic acid; polylactic acid; polydioxanone; caprolactone; polymer materials based on one or more of glycolide, l-lactide, p-dioxanone, trimethylene carbonate and ε-caprolactone; and any combination thereof. The plurality of the hooks 1011 are on the body 1010 of the first implant 101. Each of the plurality of the hooks 1011 includes a vertex and a bottom, and the bottom of the hook 1011 may be directly connected to the body 1010 of the first implant 101 or be a part of the body 1010 of the first implant 101. The hook 1011 may further includes a central axis passing through the vertex and the central of the bottom, and an angle α between the central axis of the hook 1011 and the bottom of the hook 1011 is not 90°. The plurality of hooks 1011 may be made of the same materials as the body 1010 of the first implant 101, but exemplary embodiments are not so limited. In one embodiment, the plurality of the hooks 1011 may be made of the non-absorbable materials and the body 1010 of the first implant 101 may be made of the absorbable materials. In other embodiment, the plurality of the hooks 1011 may be made of the non-absorbable materials which are different from the non-absorbable materials in the body 1010 of the first implant 101. In other embodiment, the plurality of the hooks 1011 may be made of the absorbable materials which are different from the absorbable materials in the body 1010 of the first implant 101. For example, the non-absorbable materials in the plurality of the hooks 1011 may be nylon, and the non-absorbable materials in the body 1010 may be polydioxanone, or both of the non-absorbable materials in the hooks 1011 and the body 1010 may be nylon. In other embodiment, each of the plurality of the hooks 1011 may be made of the same or different materials, for example, each of the plurality of the hooks 1011 may be made of nylon, or some of the plurality of the hooks may be made of nylon and others may be made of polybutester. In an embodiment, the plurality of the hooks 1011 may be the integrated formed with the body 1010 of the first implant 101, but exemplary embodiments are not so limited.

Referring to FIG. 4, the first implant 101′ according to the other exemplary embodiment of the present invention has a body 1010′ and a plurality of the hooks 1011′ which can engage dermis and pulling scalp in needed thereof from the sides of skull upward (i.e. pulling the scalp from one side temporal area toward to a zenith) on the body 1010′. The body 1010′ of the first implant 101′ according to an exemplary embodiment of the present invention may be a ribbon made of non-absorbable materials or absorbable materials. The example of the non-absorbable materials may include but not limit to nylon, polypropylene (Prolene), polybutester (Novafil), and any combination thereof. The example of the absorbable materials may include but not limit to catgut; polyglycolic acid; polylactic acid; polydioxanone; caprolactone; polymer materials based on one or more of glycolide, l-lactide, p-dioxanone, trimethylene carbonate and ε-caprolactone; and any combination thereof. Each of the plurality of the hooks 1011′ includes a vertex and a bottom, and the bottom of the hook 1011′ may be directly connected to the body 1010′ of the first implant 101′ or be a part of the body 1010′ of the first implant 101′. The hook 1011′ may further includes a central axis passing through the vertex and the central of the bottom, and an angle α between the central axis of the hook 1011′ and the bottom of the hook 1011′ is not 90°. The plurality of the hooks 1011′ may be made of the same materials as the body 1010 of the first implant 101′, but exemplary embodiments are not so limited. In one embodiment, the plurality of the hooks 1011′ may be made of the non-absorbable materials and the body 1010′ of the first implant 101′ may be made of the absorbable materials. In other embodiment, the plurality of the hooks 1011′ may be made of the non-absorbable materials which are different from the non-absorbable materials in the body 1010′ of the first implant 101′. In other embodiment, the plurality of the hooks 1011′ may be made of the absorbable materials which are different from the absorbable materials in the body 1010′ of the first implant 101′. For example, the non-absorbable materials in the plurality of the hooks 1011′ may be nylon, and the non-absorbable materials in the body 1010′ may be polydioxanone, or both of the non-absorbable materials in the hooks 1011′ and the body 1010′ may be nylon. In other embodiment, each of the plurality of the hooks 1011′ may be made of the same or different materials, for example, each of the plurality of the hooks 1011′ may be made of nylon, or some of the plurality of hooks may be made of nylon and others may be made of polybutester. In an embodiment, the plurality of the hooks 1011′ may be the integrated formed with the body 1010′ of the first implant 101′, but exemplary embodiments are not so limited.

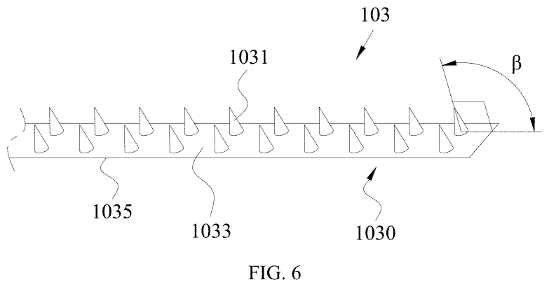

FIG. 5 is a schematic view illustrating an arrangement according to the other exemplary embodiment of the present invention and FIG. 6 is a schematic view of the third implant 103 of FIG. 5 according to an exemplary embodiment of the present invention.

The arrangement illustrated in FIG. 5 is the same as the arrangement illustrated in FIG. 1, except that the arrangement illustrated in FIG. 5 further includes embedding a third implant 103 in the subcutaneous tissue in the scalp to provide more force to pull the scalp from the temporal areas toward to the top of the head 10. Referring to FIGS. 5 and 6, the third implant 103 has a ring-shaped body 1030 and a plurality of spikes 1031 on the ring-shaped body 1035. Referring to FIG. 5, a diameter of the ring-shaped body 1035 may be substantially 10-20 cm for enclosing the top of the head 10 and intersects with the plurality of the first implants 101 and the plurality of the second implants 102 respectively.

The ring-shaped body 1030 of the third implant 103 according to an exemplary embodiment of the present invention may be a ribbon made of non-absorbable materials or absorbable materials. The example of the non-absorbable materials may include but not limit to nylon, polypropylene (Prolene), polybutester (Novafil), and any combination thereof. The example of the absorbable materials may include but not limit to catgut; polyglycolic acid; polylactic acid; polydioxanone; caprolactone; polymer materials based on one or more of glycolide, l-lactide, p-dioxanone, trimethylene carbonate and ε-caprolactone; and any combination thereof. Each of the plurality of the spikes 1031 includes a vertex and a bottom, and the bottom of the spikes 1031 may be directly connected to the ring-shaped body 1030 of the third implant 103 or be a part of the ring-shaped body 1030 of the third implant 103. The spikes 1031 may further includes a central axis passing through the vertex and the central of the bottom, and an angle β between the central axis of the spikes 1031 and the bottom of the spikes 1031 may be substantially 90°. The plurality of the spikes 1031 may be made of the same materials as the ring-shaped body 1030 of the third implant 103, but exemplary embodiments are not so limited. In one embodiment, the plurality of the spikes 1031 may be made of the non-absorbable materials and the ring-shaped body 1035 of the third implant 103 may be made of the absorbable materials. In other embodiment, the plurality of the spikes 1031 may be made of the non-absorbable materials which are different from the non-absorbable materials in the ring-shaped body 1035 of the third implant 103. In other embodiment, the plurality of the spikes 1031 may be made of the absorbable materials which are different from the absorbable materials in the ring-shaped body 1035 of the third implant 103. For example, the non-absorbable materials in the plurality of the spikes 1031 may be nylon, and the non-absorbable materials in the ring-shaped body 1035 may be polydioxanone, or both of the non-absorbable materials in the spikes 1031 and the ring-shaped body 1035 may be nylon. In other embodiment, each of the plurality of the spikes 1031 may be made of the same or different materials, for example, each of the plurality of the spikes 1031 may be made of nylon, or some of the plurality of the spikes may be made of nylon and others may be made of polybutester. In an embodiment, the plurality of the spikes 1031 may be the integrated formed with the ring-shaped body 1035 of the third implant 103, but exemplary embodiments are not so limited.

Referring to FIG. 6, the ring-shaped body 1030 according to an exemplary embodiment of the present invention has an inner face 1035 and an outer face 1033, and the plurality of the spikes 1031 are disposed on the outer face 1033, but exemplary embodiments are not so limited. In other exemplary embodiments, the ring-shaped body 1030 may further include a plurality of holes through the ring-shaped body 1030, so that the fixation and the length of the third implant could be adjusted even months after the completion of the embed operation of the third implant according to the present invention, i.e. when the scalp and the third implant has disengaged and become loose, the fixation and the length of the third implant could be adjusted by the plurality of holes in the ring-shaped body of the third implant.

The third implant 103 of FIG. 6 may be embedded after the embed operation of the plurality of the first implants 101 and the plurality of the second implants 102 is completed. The third implant 103 may be inserted into a main mini-incision above the hair line and pushed and pulled by a long and curved needle which pierces under the scalp in the subcutaneous level several times to form a round shape. As presented in FIG. 5, the third implant 103 may be embedded in a round shape in the subcutaneous tissue in the scalp, and the ring-shaped body 1030 of the third implant 103 may intersect with each of the first implants 101 and the second implants 102, so that the first implants 101, the second implants 102 and third implant 103 can keep in good close contact and move together in persistently. Therefore, the strength of the antigravity pulling force can hold for a long period of time to help the subcutaneous tissue gain enough relief of tension for regrowth of blood vessels and hair follicles. Further, a space will be created after relieving the subcutaneous space. In an embodiment, the space could be filled with growth factors by injection to stimulate the rejuvenation process.

The outer face 1033 could be kept toward the dermis layer over the subcutaneous tissue during the embed operation of the third implant 103, so that the spikes 1031 disposed on the outer face 1033 may be pointing outward for better long term integration mechanically with the scalp and the embed operation of the third implant 103 would be easier due to the smooth inner face 1035. The spikes 1031 could be engaged to a under surface of the dermis layer by pushing or squeezing. The third implant 103 will also bring the scalp upward through tightening the third implant 103 due to the ball like shaped skull.

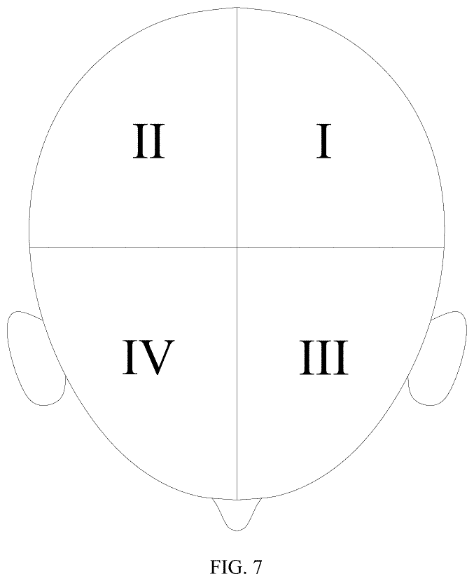

Thereinafter, the following examples of the invention are provided to show the advantages of the present invention. However, these examples are not intended to limit the scope of the one or more embodiments of the invention. It will be understood by one of ordinary skill in the art that modifications may be made to the one or more exemplary embodiments without departing from the spirit and scope of the inventive concept. The examples of the invention and comparative example are performed in four areas of a head. The four areas could be referred to as areas I-IV. Distribution of areas I-IV is present in FIG. 7.

### Example 1

3 pairs of the implants are embedded in subcutaneous tissue of areas III and IV, and spaces between two adjacent pairs of the implants are 2 cm respectively. Further, all of the pairs of the implants are in parallel with a horizontal line of the top of the head. PRP are injected into areas II and IV. The treatments performed in areas I˜IV are listed in Table 1.

TABLE 1

| Areas | Treatment |
|---|---|
| I | Non-treatment |
| II | growth factors injected |
| III | sutures embedded |
| IV | sutures embedded + growth factors injected |

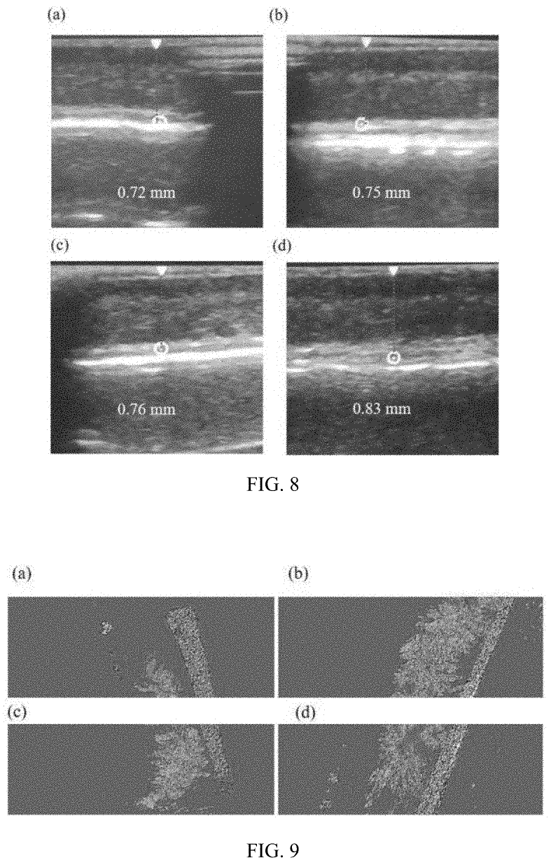

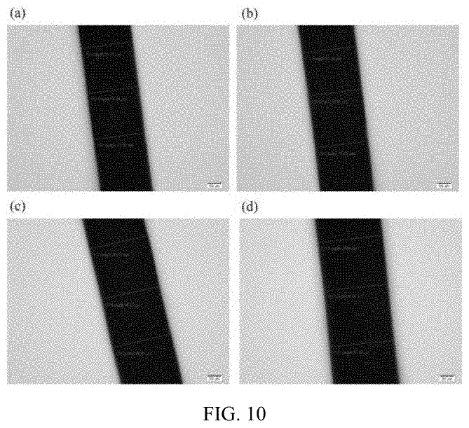

Scalp thicknesses of areas I˜IV are measured by a high-resolution ultrasound imaging system after 6 months of treatment, and result of the scalp thicknesses of areas I˜IV are shown in FIG. 8. Parts (a)-(d) of FIG. 8 are high-resolution ultrasound images of the scalp of areas I˜IV respectively. Referring to part (a) of FIG. 8, the thickness of the scalp of area I is 0.72 mm, referring to part (b) of FIG. 8, the thickness of the scalp of area II is 0.75 mm, referring to part (c) of FIG. 8, the thickness of the scalp of area III is 0.76 mm, and referring to part (d) of FIG. 8, the thickness of the scalp of area IV is 0.83 mm. Scalp blood flows of areas I˜IV are measured by a MoorFLPI full-field laser perfusion imaging system after 6 months of treatment, and result of the scalp blood flows of areas I˜IV are shown in FIG. 9. FIG. 9 is a full-field laser perfusion image of the scalp of areas I˜IV wherein part (a) of FIG. 9 is a scalp perfusion of area I, part (b) of FIG. 9 is a scalp perfusion of area II, part (c) of FIG. 9 is a scalp perfusion of area III, and part (d) of FIG. 9 is a scalp perfusion of area IV. Hair diameters of areas I˜IV are measured by an optical microscopy after 6 months of treatment, and result of the hair diameters of areas I˜IV are shown in FIG. 10. FIG. 10 is an optical microscope image of the scalp of areas I˜IV, wherein part (a) of FIG. 10 is an optical microscope image of area I, part (b) of FIG. 10 is an optical microscope image of area II, part (c) of FIG. 10 is an optical microscope image of area III, and part (d) of FIG. 10 is an optical microscope image of area IV.

Parameters of the scalp thickness, the scalp blood flow, and the hair diameter from areas I˜IV are presented in Table 2.

TABLE 2

| | Scalp thickness | | Scalp blood flow | | Hair diameter | |
|---|---|---|---|---|---|---|
| Area | (mm)(mean ± SD) | p value | (flux unit)(mean ± SD) | p value | (μm)(mean ± SD) | p value |
| I | 0.62 ± 0.09 | | 87.00 ± 17.34 | | 64.60 ± 14.08 | |
| II | 0.63 ± 0.10 | 0.182 | 101.13 ± 27.38 | 0.280 | 68.50 ± 15.28 | 0.034 |
| III | 0.65 ± 0.07 | 0.064 | 104.44 ± 24.38 | 0.075 | 70.61 ± 14.58 | 0.134 |
| IV | 0.69 ± 0.09 | 0.002 | 122.15 ± 28.34 | 0.014 | 81.21 ± 12.99 | 0.013 |

* p: Treatment different from nontreatment

Referring to Table 2 and FIGS. 8-10, it is clear that compared to the growth factors-injected treatment, the sutures-embedded treatment is more beneficial in improving the scalp thickness, the scalp blood flow, and the hair diameter. Further, the significant improvement of combining the growth factors-injected treatment and the sutures-embedded treatment is more apparent than that of using the sutures-embedded treatment only.

Therefore, it is shown that the method for treating alopecia of the present invention may have a better effect than the conventional methods for treating alopecia.

While particular embodiments of the present invention have been shown and described, it will be obvious to those skilled in the art that, based upon the teachings herein, changes and modifications may be made without departing from this invention and its broader aspects. Therefore, the appended claims are intended to encompass within their scope of all such changes and modifications as are within the true spirit and scope of the exemplary embodiment(s) of the present invention.

What is claimed is:

1. A method for treating alopecia, comprising embedding a plurality of first implants and a plurality of second implants in subcutaneous tissue of scalp in accordance with an arrangement, each of the first implant and the second implant comprising a body and a plurality of hooks on the body, wherein in the said arrangement:
directions of the plurality of first implants are from one side temporal area of a head to the top of the head;
directions of the plurality of second implants are from another side temporal area of the head to the top of the head;
the plurality of first implants and the plurality of second implants are substantially in parallel with a horizontal line of the top of the head;
each of the plurality of first implants is bonded with a corresponding second implant of the plurality of second implants at the top of the head to form a plurality pairs of implants; and
each of the plurality of hooks on the plurality of first implants and the plurality of second implants is pointing upward.

2. The method according to claim 1, wherein the body of each of the first implant and the second implant is a ribbon.

3. The method according to claim 1, wherein the body of each of the first implant and the second implant is a suture.

4. The method according to claim 1, wherein the number of the plurality pairs of implants is 4-10.

5. The method according to claim 1, wherein a space between two adjacent pairs implants of the plurality pairs of implants is substantially 1.5-2 cm.

6. The method according to claim 1, further comprising injecting growth factors into the subcutaneous tissue of the scalp.

7. The method according to claim 1, further comprising embedding a third implant in the subcutaneous tissue of the scalp, wherein the third implant has a ring-shaped body and a plurality of spikes on the ring-shaped body, and the ring-shaped body encloses the top of the head and intersects with the plurality of first implants and the plurality of second implants respectively.

8. The method according to claim 7, wherein an angle between each of the plurality of spikes and the ring-shaped body is substantially 90°.

9. The method according to claim 7, wherein a diameter of the ring-shaped body is substantially 10-20 cm.

10. The method according to claim 7, wherein the ring-shaped body is a ring-shaped ribbon.
